(19) United States
(12) Patent Application Publication
(10) Pub. No.: US 2016/0129066 A1
(43) Pub. Date: May 12, 2016

(54) ALL NATURAL TOPICAL PAIN RELIEF CREAM

(71) Applicants: Mario Medri, Newark, NJ (US); Judith Shewatjon, Brooklyn, NY (US)

(72) Inventors: Mario Medri, Newark, NJ (US); Judith Shewatjon, Brooklyn, NY (US)

(21) Appl. No.: 14/538,077

(22) Filed: Nov. 11, 2014

(51) Int. Cl. A61K 36/82; A61K 36/61; A61K 36/30; A61K 31/618; A61K 31/7034; A61K 31/05; A61K 31/125; A61K 9/06; A61K 36/185

(52) U.S. Cl. CPC A61K 36/82; A61K 9/06; A61K 36/61; A61K 36/30; A61K 36/185; A61K 31/7034; A61K 31/05; A61K 31/125; A61K 31/618

(57) ABSTRACT

The present invention provides an all-natural, topical pain relief cream, with no artificial ingredients using Bentonite U.S.P. and sterile water U.S.P. as the foundation. The cream contains as active ingredients, one or more of menthol (regular or maximum strength), and salicin (methyl salicylate), with camphor and capsaicin as optional active ingredients. The vehicle includes coconut oil and lecithin to help retain the more volatile ingredients in with cream.

### FIELD OF THE INVENTION

This invention relates to topical pain relief cream, and more particularly to a pain relief cream having all natural ingredients that can be applied topically for local relief in pain and inflammation in muscles, bones, and joints, caused by injury or arthritis.

### BACKGROUND OF THE INVENTION

Numerous topical creams, ointments, and lotions exist for treatment and relief of muscle, bones, and joint pain and inflammation, caused by injury, accident, overuse, over-extension or a disease such as arthritis, whether temporary or chronic. Such products often contain wintergreen, menthol, or aspirin, which when absorbed through skin may provide temporary relief of symptoms, and may help to promote healing. Both wintergreen and menthol have strong odors that some users may find objectionable. In addition, many patients having muscle or joint paint or inflammation prefer a treatment that uses natural and organic ingredients, and avoids synthetic ingredients, such as aspirin. A need exists therefore for a pain relief cream that avoids use of artificial ingredients and also is pleasant feeling when applied, easily absorbed, effective in relieving pain, and pleasant smelling.

### SUMMARY OF THE INVENTION

This and other objectives are achieved and the disadvantages of previously formulated or available pain relief creams are overcome with the present invention, which provides: A pain relief cream comprising: about 10% to about 20% Bentonites; about 1% to about 5% clove oil concentrate; about 10% to about 20% confrey concentrate; about 10% to about 20% nettles concentrate; about 0% to about 15% salicin (methyl salicylate); about 10% to about 20% coconut oil; about 0.5% to about 2% menthol; about 0.5% to about 2% green tea extract concentrate; about 0.1% to about 0.5% Linatural NLP-O (preservative); about 1.0% to about 3.0% lecithin; and about 20% to about 30% water (U.S.P.) to make 100%.

The invention further provides a method for relieving pain using the topical cream of the present invention. Pain relief can be accomplished without resort to oral medications or to artificially synthesized medications, such as aspirin, acetaminophen, or ibuprofen, which may have side effects or be poorly tolerated by some patients.

### DETAILED DESCRIPTION OF THE PREFERRED EMBODIMENTS

The all natural pain relief cream is of the present invention topically applied directly to sites affected by localized inflammation of muscles, bone joints and arthritic conditions. This cream is a composition of botanical extract of different plants and specific parts of plants that are known of having pain relief properties. The combination of menthol, approved by the US-FDA as a topical pain relief ingredient, to the listed and effective botanical extracts and vegetable fats all embodied into the highly absorbent, natural, and pharmaceutically acceptable Bentonite (U.S.P.), a naturally occurring clay to produce a gentle cream that when topically applied to aching parts of the body produces a very quick sensation of relief.

Botanical extracts recognized as fluid extracts of different plants such as nettle, camry, white willow bark (also known as weeping willow), and of its fluid extract is a source of Salicin (the precursor of Aspirin). These may be combined with oil of clove, menthol and other ingredient like vitamin E and Beta-Carotene creates a mixture of natural botanical extracts with significant pain relief properties.

### Composition

The product is identified as a typical cream with a non-flowing yet spreadable texture, which when applied to any portion of a human skin disperses in a very thin layer permanently absorbing of its components. The consistency of the cream is based on the combination of the botanical extracts, oils, water and emulsifiers, which are all absorbed onto the Bentonite (U.S.P.) a naturally occurring clay. Formulations are differentiated by the amount of menthol, which is a therapeutically recognized pain relief active ingredient. Formulations are identified as “Regular Strength” (about 0.5% menthol), “Maximum Strength” (about 1.0% menthol) and “Maximum Strength with Salicin (about 1.0% menthol).” The amount of menthol present in the formulations are the amounts recognized by the FDA as effective and listed in the Drug Monograph. Formulations of commercially available products contain 5% to 10% of menthol, which exceeds FDA recommended dosages. Salicin, a natural fluid extract delivers an effective natural dose of methyl salicylate. Salicin is not used in commercial products even though many clinical studies have demonstrated of its effectiveness to be comparable to the synthetic version of it as methyl salicylate. Methyl Salicylate is an effective anti-inflammatory compound and listed in the FDA Drug monograph as such. Salicin as methyl salicylate can optionally be used in formulating the present invention from 5 to 15 percent by weight, with about 12% preferred. Camphor also has pain relieving qualities, and can be added in an amount of 0.5% to about 2.0% by weight. Capaxin, another active ingredient that stimulates blood to the affected area, can be added in an amount of about 2% to about 5%, or as suggested by FDA monograph of approved dosages, giving thought to its potential to irritate a user's skin. Clove oil can also be used in an amount ranging from about 2% to about 5% while nettles and confrey, two herbal remedies can be provided in amount ranging from about 10% to about 15% by weight. The inactive ingredients that helped to make a pleasant feeling and smelling cream include Bentonite (powdered clay) U.S.P., in an amount ranging from about 15% to about 20 or even 25% depending on the particular active ingredients used, and the amount of liquid that needs to be absorbed to form the proper cream consistency. The same is true of coconut oil, lecithin, and water, all of which may be varied in formulation by a person of ordinary skill to achieve a thicker or thinner consistency, as desired for particular application.

The following are examples of formulations reflecting the natural pain relief creams where Menthol is the sole active ingredient compliant with the US-FDA drug Monograph. These examples are meant to be illustrative of the practices of the invention, but not limiting.

### Example 1

### Pain Relief Cream—Regular Strength with Menthol

| | |
|---|---|
| | Menthol |
| | Regular Strength |
| PRODUCT | Lot No. #04252014-R |

| Regular Strength | | Pilot Batch |
|---|---|---|
| Formula | % | (gm.) |
| Powdered Clay (Bentonite U.S.P.) | 17.663 | 76.00 |
| Clove Oil Conc. (FFS RF18) | 3.512 | 15.11 |
| Confrey Conc. 1:3 | 14.765 | 63.53 |
| Nettles Conc. 1:3 | 14.765 | 63.53 |
| Coconut Oil | 14.765 | 63.53 |
| Menthol FFS (RD230) (U.S.P.) | 0.581 | 2.50 |
| Water (U.S.P.) | 30.795 | 132.50 |
| Green Tea Conc. FFS 53784 | 1.046 | 4.50 |
| Linatural NLP-O (preservative) | 0.351 | 1.51 |
| Lecithin | 1.757 | 7.56 |
| Total | 100.000 | 430.27 gm. |

### Example 2

### Pain Relief Cream—Maximum Strength with Menthol

| | |
|---|---|
| | Menthol |
| | Maximum Strength |
| PRODUCT | Lot No. #04252014-M |

| Maximum Strength | | Pilot Batch |
|---|---|---|
| Formula | % | (gm.) |
| Powdered Clay (Bentonite U.S.P.) | 17.561 | 76.00 |
| Clove Oil Conc. (FFS RF18) | 3.491 | 15.11 |
| Confrey Conc. 1:3 | 14.680 | 63.53 |
| Nettles Conc. 1:3 | 14.680 | 63.53 |
| Coconut Oil | 14.680 | 63.53 |
| Menthol FFS (RD230) (U.S.P.) | 1.155 | 5.00 |
| Water (U.S.P.) | 30.617 | 132.50 |
| Green Tea Conc. FFS 53784 | 1.040 | 4.50 |
| Linatural NLP-O (preserv.) | 0.349 | 1.51 |
| Lecithin | 1.747 | 7.56 |
| Total | 100.000 | 432.77 gm. |

### Process:

The following is the process being used for the formula preparations listed above.

### Primary Blend

In a dry heat oven place the powder clay on a tray and keep it at 130-140° C. for 30-40 minutes, with the intent of to sterilize the clay. Than, boil the U.S.P. water for 20 minutes, with the intent to sterilize the water, and to the boiling water add the still hot powder clay. Mix the clay into the water to assure proper distribution and complete wetness of the clay particles. Adjust the amount of water as per formula requirement.

When the clay is well dispersed, add to the clay water mixture all the fluid extracts such as Confrey and Nettles, and mix well to assure complete dispersion. The botanical fluid extracts are all composed of natural products such as water and ethyl alcohol.

### Secondary Blend

In a separate container liquefy coconut oil by brining the oil to a temperature of about 50° C. Than add Lecithin to the oil and disperse it very well while keeping the oil at 50° C. When very well blended, add Menthol, and Oil of Clove and mix very quickly to reduce the sublimation of menthol. When all done than add the secondary blend to the Primary Blend and mix for 10 minutes to get a very uniform cream. Add Preservative and Fragrance and mix for 10 minutes to assure proper ingredient distribution. Transfer the cream to filling machine while keeping the mass at 50° C.

The following are further exemplary formulations reflecting the natural pain relief cream of the present invention where menthol is the primary active ingredient in dosages complying with the US-FDA Drug Monograph. In addition, secondary active ingredients like salicin, camphor, methyl salicylate and capsaicin are added in different ratios and combinations thereof.

### Example 3

### Pain Relief Cream—Maximum Strength with Menthol and Salicin (Methyl Salicylate)

| | |
|---|---|
| | Menthol & Salicin |
| | Maximum Strength |
| PRODUCTS | Lot No. #04252014-MS |

| Maximum Strength | | Pilot Batch |
|---|---|---|
| Formula | % | (gm.) |
| Powder Clay (Bentonite U.S.P.) | 17.383 | 76.00 |
| Clove Oil Conc. (FFS RF18) | 3.456 | 15.11 |
| Confrey Conc. 1:3 | 13.495 | 59.00 |
| Nettles Conc. 1:3 | 13.495 | 59.00 |
| White Willow Bark Conc. 1:3 | 10.293 | 45.00 |
| Coconut Oil | 14.531 | 63.53 |
| Menthol FFS (RD230) (U.S.P.) | 1.029 | 4.50 |
| Water (U.S.P.) | 23.215 | 101.50 |
| Green Tea Conc. FFS 53784 | 1.029 | 4.50 |
| Linatural NLP-O (preservative) | 0.345 | 1.51 |
| Lecithin | 1.729 | 7.56 |
| Total | 100.00 | 437.21 gm. |

### Process:

The following is the process being used for the formula preparation listed above.

### Primary Blend

In a dry heat oven place the powdered clay on a tray and keep it at 130-140° C. for 30-40 minutes to sterilize the clay. Than, boil the U.S.P. water for 20 minutes to sterilize the water. Add the still hot powdered clay to the boiling water. Mix the clay into the water to assure proper distribution and complete wetness of the clay particles. Adjust the amount of water as per formula requirements.

When the clay is well-dispersed, add all of the fluid extracts such as confrey, nettles, and salicin to the clay and water mixture and mix well to assure complete dispersion. The botanical fluid extracts are all dissolved or suspended in natural products such as water and ethyl alcohol.

### Secondary Blend

In a separate container, liquefy coconut oil by bringing the oil to a temperature of about 50° C. Then add lecithin to the oil and disperse it very well while keeping the oil at 50° C. When very well blended, add menthol, and oil of clove and mix very quickly to reduce the sublimation of menthol. Next, add the secondary blend to the primary blend and mix for 10 minutes to obtain a very uniform cream. Add Preservative and Fragrance and mix for 10 minutes to assure proper ingredient distribution. Transfer the cream to filling machine while keeping the mass at 50° C.

### Example 4

### Pain Relief Cream—Maximum Strength with Menthol—Methyl Salicylate

| | Menthol & Methyl Salicylate | |
|---|---|---|
| | Maximum Strength | |
| PRODUCTS | Lot No. #04252014-MS | |
| Maximum Strength | | Pilot Batch |
| Formula | % | (gm.) |
| Powdered Clay (Bentonite U.S.P.) | 17.403 | 76.00 |
| Clove Oil Conc. (FFS RF18) | 3.460 | 15.11 |
| Confrey Conc. 1:3 | 13.510 | 59.00 |
| Nettles Conc. 1:3 | 13.510 | 59.00 |
| Methyl Salicylate | 10.190 | 44.50 |
| Coconut Oil | 14.547 | 63.53 |
| Menthol FFS (RD230) (U.S.P.) | 1.030 | 4.50 |
| Water (U.S.P.) | 23.242 | 101.50 |
| Green Tea Conc. FFS 53784 | 1.030 | 4.50 |
| Linatural NLP-O (preservative) | 0.346 | 1.51 |
| Lecithin | 1.731 | 7.56 |
| Total | 100.00 | 436.71 gm. |

### Process:

The following is the process being used for the formula preparation listed above.

### Primary Blend

In a dry heat oven place the powdered clay on a tray and keep it at 130-140° C. for 30-40 minutes to sterilize the clay. Then, boil the U.S.P. water for 20 minutes to sterilize the water, and add the still hot powdered clay to the boiling water. Mix the clay into the water to assure proper distribution and complete wetness of the clay particles. Adjust the amount of water as per formula requirements.

When the clay is well-dispersed, add to the clay and water mixture all of the fluid extracts such as confrey and nettles, and mix well to assure complete dispersion. The botanical fluid extracts are all composed of natural products such as water and ethyl alcohol.

### Secondary Blend

In a separate container, liquefy the coconut oil by bringing the oil to a temperature of about 50° C. Then add lecithin to the oil and disperse it very well while keeping the oil at 50° C. When very well blended, add menthol, methyl salicylate and oil of clove and mix very quickly to reduce the sublimation of menthol. Next, add the secondary blend to the primary blend and mix for 10 minutes to get a very uniform cream. Add Preservative and Fragrance and mix for 10 minutes to assure proper ingredient distribution. Transfer the cream to filling machine while keeping the mass at 50° C.

### Example 5

### Pain Relief Cream—Maximum Strength with Menthol—Methyl Salicylate and Camphor

| | Menthol & Methyl Salicylate | |
|---|---|---|
| | Maximum Strength | |
| PRODUCTS | Lot No. #04252014-MS | |
| Maximum Strength | | Pilot Batch |
| Formula | % | (gm.) |
| Powdered Clay (Bentonite U.S.P.) | 16.840 | 76.00 |
| Clove Oil Conc. (FFS RF18) | 3.348 | 15.11 |
| Confrey Conc. 1:3 | 13.073 | 59.00 |
| Nettles Conc. 1:3 | 13.073 | 59.00 |
| Methyl Salicylate | 10.060 | 45.40 |
| Camphor | 3.013 | 13.60 |
| Coconut Oil | 14.077 | 63.53 |
| Menthol FFS (RD230) (U.S.P.) | 1.019 | 4.60 |
| Water (U.S.P.) | 22.490 | 101.50 |
| Green Tea Conc. FFS 53784 | 0.997 | 4.50 |
| Linatural NLP-O (preservative) | 0.335 | 1.51 |
| Lecithin | 1.675 | 7.56 |
| Total | 100.000 | 451.31 gm. |

### Process:

The following is the process being used for the formula preparation listed above.

### Primary Blend

In a dry heat oven place the powdered clay on a tray and keep it at 130-140° C. for 30-40 minutes to sterilize the clay. Then boil the U.S.P. water for 20 minutes to sterilize the water, and add the still hot powdered clay to the boiling water. Mix the clay into the water to assure proper distribution and complete wetness of the clay particles. Adjust the amount of water as per formula requirement.

When the clay is well dispersed, add all the fluid extracts such as Confrey and Nettles to the clay and water mixture, and mix well to assure complete dispersion. The botanical fluid extracts are all suspended or dissolved of natural products such as water and ethyl alcohol.

### Secondary Blend

In a separate container, liquefy coconut oil by brining the oil to a temperature of about 50° C. Then add lecithin to the oil and disperse it very well, while keeping the oil at 50° C. When very well blended, add menthol, camphor, methyl salicylate, oil of clove and mix very quickly to reduce the sublimation of the menthol and camphor. When thoroughly mixed, add the secondary blend to the primary blend and mix for 10 minutes to get a very uniform cream. Add preservative and fragrance and mix for 10 minutes to assure proper ingredient distribution. Transfer the cream to filling machine while keeping the mass at 50′C.

### Example 6

### Pain Relief Cream—Maximum Strength with Menthol—Methyl Salicylate—Camphor and Capsaicin

| | Menthol & Methyl Salicylate | |
|---|---|---|
| | Maximum Strength | |
| PRODUCTS | Lot No. #04252014-MS | |
| Maximum Strength | | Pilot Batch |
| Formula | % | (gm.) |
| Powdered Clay (Bentonite U.S.P.) | 16.482 | 76.00 |
| Clove Oil Conc. (FFS RF18) | 3.277 | 15.11 |
| Confrey Conc. 1:3 | 12.795 | 59.00 |
| Nettles Conc. 1:3 | 12.795 | 59.00 |
| Methyl Salicylate | 9.759 | 45.00 |
| Camphor | 2.060 | 9.50 |
| Capsaicin | 3.080 | 14.20 |
| Coconut Oil | 13.778 | 63.53 |
| Menthol FFS (RD230) (U.S.P.) | 1.019 | 4.70 |
| Water (U.S.P.) | 22.012 | 101.50 |
| Green Tea Conc. FFS 53784 | 0.976 | 4.50 |
| Linatural NLP-O (preservative) | 0.327 | 1.51 |
| Lecithin | 1.640 | 7.56 |
| Total | 100.000 | 461.11 gm. |

### Process:

The following is the process being used for the formula preparation listed above.

### Primary Blend

In a dry heat oven place the powdered clay on a tray and keep it at 130-140° C. for 30-40 minutes to sterilize the clay. Then boil the water (U.S.P.) for 20 minutes to sterilize the water, and add the still hot powdered clay to the boiling water. Mix the clay into the water to assure proper distribution and complete wetness of the clay particles. Adjust the amount of water as per formula requirements.

When the clay is well-dispersed, add all the fluid extracts such as confrey and nettles to the clay mixture and mix well to assure complete dispersion. The botanical fluid extracts are all suspend or dissolved in natural products such as water and ethyl alcohol.

### Secondary Blend

In a separate container liquefy coconut oil by bringing the oil to a temperature of about 50° C. Than add Lecithin to the oil and disperse it very well while keeping the oil at 50° C. When very well blended, add menthol, oil of clove, camphor, methyl salicylate and capsaicin and mix very quickly to reduce the sublimation of menthol and of camphor. When all done, then add the secondary blend to the primary blend and mix for 10 minutes to get a uniform cream.

Add preservative and fragrance and mix for 10 minutes to assure proper ingredient distribution. Transfer the cream to filling machine while keeping the mass at 50° C.

### DISCUSSION

The pain relief product of the present invention is 100% natural and based on several active ingredients (menthol, camphor, and salicin (methyl salicylate)) that are approved and recognized by the US-FDA as effective as topical pain relieving ingredients individually and in combination. Such active ingredients are listed in the FDA Monograph as topical remedies. In addition, these pain relief formulations are composed of all natural ingredients and are combined with botanical extracts to further assist in the relief of pain, be it of rheumatic or of muscular origin. All of these pain relief product formulations reflect a blend of natural ingredients in their composition and natural extracts assuring the patient of a usage of a natural remedy.

This product is novel in its composition and preparation especially in the combination of the clay with botanical extracts. A specific preparation method has to be followed as described to have maximum texture and maximum dispersion on the effected area. The addition and use of active ingredients of the type listed increases the sensation of relief. The botanical and organic nature of the vehicle composed of all natural extracts and will assure effectiveness in the relief of topical localized pain. This pain relief product works for adults and children as a convenient and novel natural preparations.

Products currently on the market place are not of natural origin in their composition and some claim just active ingredients of natural source. Most of the delivery vehicles are of synthetic composition, unlike the present invention, which uses natural ingredients for both the active ingredients and the vehicle.

Ingredients like clay, menthol, camphor, methyl salicylate and botanical extracts are all of natural origin and found to be effective, individually or in combination of, in the reduction of arthritic and muscle pain. Some of the ingredients are listed in the FDA's drug monograph and using them in their natural state is very important in order to maintain a natural cream composition. Botanical extracts valuable adjuncts to the monograph ingredients rendering the pain cream of the present invention more effective in achieving relief. The cream is found to be effective in pain management.

1. A pain relief cream comprising:
about 10% to about 20% Bentonite (U.S.P.);
about 1% to about 5% clove oil concentrate;
about 10% to about 20% confrey concentrate;
about 10% to about 20% nettles concentrate;
about 0% to about 15% salicin (methyl salicylate);
about 10% to about 20% coconut oil;
about 0.5% to about 2% menthol;
about 0.5% to about 2% green tea extract concentrate;
about 0.1% to about 0.5% Linatural NLP-O (preservative);
about 1.0% to about 3.0% lecithin; and
about 20% to about 30% sterile water (U.S.P.) to make 100%.

2. A pain relief cream according to claim 1, additionally comprising:
about 5% to about 15% salicin as methyl salicylate.

3. A pain relief cream according to claim 2, additionally comprising about 1% to about 3% camphor.

4. A pain relief cream according to claim 2, wherein the amount of menthol is between about 0.5% and about 1%.

5. A pain relief cream according to claim 4, wherein the amount of salicin or methyl salicylate is about 8% to about 12%.

6. A topical pain relief cream according to claim 5, wherein the amount of salicin is about 10% by weight, the amount of camphor is about 2% by weight; the amount of menthol is about 1% by weight; and the amount of Bentonite or clay is about 15% by weight to about 19% by weight.

7. A method for treating a painful region comprising applying in patient painful region an effective amount of the pain cream as set forth in claim 1.

8. A method for treating a painful region comprising applying in patient painful region an effective amount of the pain cream as set forth in claim 2.

9. A method for treating a painful region comprising applying in patient painful region an effective amount of the pain cream as set forth in claim 3.

10. A method for treating a painful region comprising applying in patient painful region an effective amount of the pain cream as set forth in claim 4.

11. A method for treating a painful region comprising applying in patient painful region an effective amount of the pain cream as set forth in claim 5.

12. A method for treating a painful region comprising applying in patient painful region an effective amount of the pain cream as set forth in claim 6.
